(19) United States
(12) Patent Application Publication
(10) Pub. No.: US 2003/0008929 A1
(43) Pub. Date: Jan. 9, 2003

(54) Surface active agent use in catalyst preparation

(75) Inventor: Joe D. Allison, Ponca City, OK (US)

Correspondence Address: DAVID W. WESTPHAL, CONOCO PHILLIPS, P.O. BOX 4783, HOUSTON, TX, 77210-4783, US

(73) Assignee: Conoco Inc., Houston, TX (US)

(21) Appl. No.: 10/160,730

(22) Filed: May 31, 2002

Related U.S. Application Data
(60) Provisional application No. 60/302,776, filed on Jul. 3, 2001.

(51) Int. Cl. C07C 27/06

(52) U.S. Cl. 518/715; 518/716

(57) ABSTRACT

A method a making a catalyst, preferably a Fischer-Tropsch catalyst, includes the use of a surfactant. The surfactant is preferably a non-ionic surfactant, or alternatively, a cationic surfactant. The catalyst includes support material and catalyst material. The catalyst material preferably includes at least one Fischer-Tropsch metal, more preferably cobalt. The surfactant is preferably added to a solution containing a catalyst material in an amount sufficient to improve a measure of the activity of a catalyst containing the catalyst material, such as the CO conversion, the methane selectivity, the C₅₊ productivity, or catalyst life. A method for producing hydrocarbons includes contacting a catalyst made as described above with hydrogen and carbon monoxide.

### CROSS-REFERENCE TO RELATED APPLICATIONS

This application claims the benefit of priority of 35 U.S.C. 111(b) provisional application Serial No. 60/302,776 filed Jul. 3, 2001, and entitled “Surface Active Agent Use in Catalyst Preparation”.

### STATEMENT REGARDING FEDERALLY SPONSORED RESEARCH OR DEVELOPMENT

Not Applicable.

### FIELD OF THE INVENTION

The present invention relates generally to a method for making a catalyst. More particularly, the present invention relates to the use of a surface active agent in the preparation of a catalyst, preferably a Fischer-Tropsch catalyst, more preferably a cobalt-containing Fischer-Tropsch catalyst. Still more particularly, the present invention relates to the use of a wetting agent for the improvement of dispersion of catalytically active metal, preferably a Fischer-Tropsch metal, on a catalyst support.

### BACKGROUND OF THE INVENTION

Large quantities of methane, the main component of natural gas, are available in many areas of the world, and natural gas reserves are predicted to outlast oil reserves by a significant margin. However, most natural gas is situated in areas that are geographically remote from population and industrial centers. The costs of compression, transportation, and storage make its use economically unattractive. To improve the economics of natural gas use, much research has focused on the use of methane as a starting material for the production of higher hydrocarbons and hydrocarbon liquids, which are more easily transported and thus more economical. The conversion of methane to hydrocarbons is typically carried out in two steps. In the first step, methane is converted into a mixture of carbon monoxide and hydrogen (i.e., synthesis gas or syngas). In a second step, the syngas is converted into hydrocarbons.

This second step, the preparation of hydrocarbons from synthesis gas, is well known in the art and is usually referred to as Fischer-Tropsch synthesis, the Fischer-Tropsch process, or Fischer-Tropsch reaction(s). Fischer-Tropsch synthesis generally entails contacting a stream of synthesis gas with a catalyst under temperature and pressure conditions that allow the synthesis gas to react and form hydrocarbons.

More specifically, the Fischer-Tropsch reaction is the catalytic hydrogenation of carbon monoxide to produce any of a variety of products ranging from methane to higher alkanes and aliphatic alcohols. The product of a Fischer-Tropsch reaction is typically a mixture of hydrocarbons with different carbon chain lengths, and thus molecular weights. The range of hydrocarbon weights in the mixture can be influenced by the choice of catalyst, as well as the reactor and the reactor conditions Thus, research continues on the development of more efficient Fischer-Tropsch catalyst systems and reaction systems that increase the selectivity for high-value hydrocarbons in the Fischer-Tropsch product stream.

It is particularly desirable to maximize the production of high-value liquid hydrocarbons. Under standard conditions of temperature and pressure, liquid hydrocarbons typically include hydrocarbons with five or more carbon atoms per hydrocarbon chain (C₅₊). Desirable Fischer-Tropsch product mixtures include, for example, those containing wide range naphtha fractions, such as fractions containing C₅-C₁₂ hydrocarbons, and those containing gasoil fractions, such as fractions containing C₁₃-C₂₀ hydrocarbons. Naphtha fractions can be processed to yield gasoline, whereas gasoil fractions can be processed to yield diesel oil.

There are continuing efforts to find catalysts that are more effective at producing these desired products. Product distribution, product selectivity, and reactor productivity depend heavily on the type and structure of the catalyst and on the reactor type and operating conditions. A number of studies describe the behavior of various Fischer-Tropsch catalysts in various reactor types, together with the development of catalyst compositions and preparations. For example, see the articles “Short history and present trends of Fischer-Tropsch synthesis,” by H. Schlutz, Applied Catalysis A 186, 3-12, 1999, and “Status and future opportunities for conversion of synthesis gas to liquid fuels, by G. Alex Mills, Fuel 73, 1243-1279, 1994, each hereby incorporated herein by reference in their entirety.

Catalysts for use in the Fischer-Tropsch synthesis usually contain a catalytically active metal of Groups 8, 9, 10 (in the New notation of the periodic table of the elements, which is followed throughout), also termed herein a Fischer-Tropsch metal. In particular, iron, cobalt, nickel, and ruthenium have been abundantly used as the catalytically active metals. Nickel is useful for a process in which methane is a desired product. Iron has the advantage of being readily available. Ruthenium has the advantage of high activity but is relatively expensive and thus is typically used as a promoter for another of the Fischer-Tropsch metals. Cobalt has the advantages of being more active than iron and more available than ruthenium and less selective to methane than nickel.

Thus, cobalt has been investigated as a catalyst for the production of hydrocarbons with weights corresponding to the range of the gasoline, diesel, and higher weight fractions of crude oil. In particular, cobalt has been found to be suitable for catalyzing a process in which synthesis gas is converted to primarily hydrocarbons having five or more carbon atoms (i.e., where the C₅₊selectivity of the catalyst is high).

Additionally, catalysts often contain one or more promoters. Promoters typically depend on the identity of the catalytically active metal. For example, alkali metal promoters (e.g. potassium) are known for iron-containing Fischer-Tropsch catalysts. Further, promoters that have been used for cobalt-ruthenium catalysts include thorium, lanthanum, magnesium, manganese, and rhenium. A promoter may have any of various desirable functions, such as improving activity, productivity, selectivity, lifetime, regenerability, or other properties of catalysts and catalytic processes.

Catalysts conventionally include a support or carrier material. Supports for catalysts used in Fischer-Tropsch synthesis of hydrocarbons have typically been refractory oxides (e.g., silica, alumina, titania, zirconia or mixtures thereof, such as silica-alumina). A support may be used to reduce the amount of catalytically active metal used, to provide a high surface area for contact of the catalytically active metal with the syngas, or to otherwise improve the performance or economics of catalysts and catalytic processes.

Typically, preparation of a supported catalyst involves deposition of the catalytically active metal on the support. In one method, metal oxides or hydroxides are co-precipitated from an aqueous solution by adding a precipitating agent. In another method, metal salts are mixed with a wet support in a suitable blender to obtain a substantially homogeneous solution. Still another method that has been employed is known as incipient wetness impregnation. In this method, metal salts are dissolved in an amount of a suitable solvent just sufficient to fill the pores of the support.

In the preparation of a catalyst, it is desirable to increase dispersion of the catalyst materials, such as the catalytically active metal and any promoters, on the support. If the dispersion is low, the catalyst materials are unevenly deposited on the support, with some areas of the catalyst having higher local concentrations of catalyst materials than other areas. This has the disadvantage of inefficient utilization of catalyst materials. Further, catalyst metals such as cobalt and, in particular, rare promoters, such as ruthenium and rhenium, tend to be costly. Thus, increased catalyst metal dispersion is desirable. In particular, methods of depositing catalyst materials and making catalysts with improved productivity to C₅₊hydrocarbons are desirable.

Hence, it continues to be desirable to improve the activity of Fischer-Tropsch catalysts. In particular, methods of making catalysts are desirable which will improve catalyst activity and reduce the cost of effective catalysts.

### SUMMARY OF THE INVENTION

The present invention features the use of a surfactant, preferably a non-ionic surfactant, in the preparation of a supported catalyst, preferably a Fischer-Tropsch catalyst.

According to one embodiment of the present invention, a method for making a catalyst includes contacting a solution containing a catalyst material with a support material in the presence of a surfactant.

The catalyst material may include a Fischer-Tropsch metal. The catalyst material preferably includes a Fischer-Tropsch metal in an amount catalytically active for a Fischer-Tropsch reaction. The Fischer-Tropsch metal preferably includes cobalt, more preferably in an amount sufficient to provide a weight ratio of cobalt to support of from about 1:100 to about 1:2, more preferably from about 1:10 to about 1:3.

The surfactant is preferably a non-ionic surfactant. Suitable non-ionic surfactants include polyoxyethylenated alkyphenols, polyoxyethylenated alkyphenol ethoxylates, and the like.

Alternatively, the surfactant may be a cationic surfactant. Suitable cationic surfactants include quarternary long-chain organic amine salts, quarternary polyethoxylated long-chain organic amine salts, and the like.

According to an alternative embodiment of the present invention, a method for making a catalyst from a support material and a catalyst material includes adding a surfactant to a catalyst preparation mixture in an amount sufficient to improve a measure of the activity of the resulting supported catalyst. The measure of the activity is preferably increased by at least 5%, more preferably by at least 20%, and most preferably by at least 30%. Suitable measures of activity include the CO conversion, the methane selectivity, the C₅₊productivity, the catalyst life, and the like. The catalyst preparation mixture preferably includes a support material and a catalyst material.

The catalyst made by a method according to any of the above-described embodiments of the present invention may be used in a method for producing hydrocarbons that includes contacting a feed stream comprising hydrogen and carbon monoxide with the catalyst so as to produce an effluent stream comprising hydrocarbons.

Thus, the present invention comprises a combination of features and advantages which enable it to overcome various problems of prior devices. The various characteristics described above, as well as other features, will be readily apparent to those skilled in the art upon reading the following detailed description of the preferred embodiments of the invention.

### DETAILED DESCRIPTION OF THE PREFERRED EMBODIMENT

According to a preferred embodiment of the present invention, a method for making a catalyst includes contacting a support with a precursor mixture containing a catalyst material in the presence of a surface active agent, also termed a surfactant.

The surfactant preferably operates to lower the surface tension at the interface between the support material and the liquid mixture containing the catalyst material. The liquid mixture is preferably in the form of a solution, more preferably an aqueous solution. Thus, the surfactant is preferably a wetting agent, effective for improving wetting of a solution containing catalyst material on the support. The wetting agent preferably has an affinity for the support and an affinity for the solution that contains catalyst material.

Alternatively or in combination, the surfactant preferably operates to lower the surface tension at the interface between a catalyst material in suspension, such as in the form of droplets, and the solvent in which the catalyst material is suspended. Thus, the surfactant is preferably a dispersing agent for the catalyst material, effective for improving dispersion of the catalyst material in the solution. The dispersing agent preferably has an affinity for the catalyst material and an affinity for the solvent.

Further, the surfactant is preferably any suitable surfactant whose use in making a catalyst favorably improves an analytical characteristic of the catalyst, such as catalyst dispersion, metal surface area, and the like.

Alternatively or in combination, the surfactant is preferably any suitable surfactant whose use in making a catalyst improves a performance characteristic of the catalyst in the Fischer-Tropsch reaction, such as C₅₊productivity, methane selectivity, CO conversion, or catalyst life.

Suitable surfactants that are contemplated for use in the preparation of catalysts containing a Fischer-Tropsch metal on refractory support include cationic and non-ionic surfactants, such as those taught in Surfactants and Interfacial Phenomena, pp. 5-24, by Milton J. Rosen, hereby incorporated herein by reference.

In particular, suitable non-ionic surfactants include, but are not limited to, polyoxyethylated alkyphenols and polyoxyethylated alkyphenol ethoxylates. For example, polyoxyethylene (10) isooctylphenyl ether is a suitable non-ionic surfactant. The use of this surfactant is described in more detail in Example 1 herein.

Alternatively, suitable cationic surfactants include, but are not limited to, quarternary long-chain organic amine salts, quarternary polyethoxylated long-chain organic amine salts.

Contacting of the mixture containing catalyst materials and surfactant with a support may be accomplished by any of the conventional methods known to those skilled in the art for contacting a catalyst material with a support utilizing a solvent. The catalyst materials in the mixture may include catalytically active metal in the form of compounds of the metal that serve as precursors. By way of illustration and not limitation, conventional methods for preparing supported catalysts include impregnating precursors onto a support, and/or precipitating the precursors onto a support.

The most preferred method for preparation may vary among those skilled in the art, depending for example on the desired catalyst particle size. Those skilled in the art are able to select the most suitable method for a given set of requirements.

One method for preparing a supported metal catalyst (e.g., a supported cobalt-containing catalyst) is by incipient wetness impregnation of the support with an aqueous solution of a soluble metal salt such as nitrate, acetate, acetylacetonate or the like. The surfactant may be added to the aqueous solution. Other conventional methods of preparing a supported metal catalyst that are adapted to the use of a surfactant include vacuum impregnation, co-precipitation, or spray dying.

The most preferred sequence of addition of elements to a support may vary among those skilled in the art. For example, it is contemplated that the Fischer-Tropsch metal and any optional promoter may be added to a support, for example by impregnation of the support, in one step. Thus a supported catalyst according to a preferred embodiment of the present invention may include co-dispersed Fischer-Tropsch metal and promoter. Alternatively, the Fischer-Tropsch metal and any optional promoter may be added to a support, for example by impregnation of the support, in separate steps. Thus, a supported catalyst according to a preferred embodiment of the present invention may include a layer containing a Fischer-Tropsch metal and a layer containing promoter. When the Fischer-Tropsch metal and any optional promoter are added in separate steps, a surfactant may be used as described according to any of the embodiment of the present invention described herein, in either one or both of those steps.

The impregnated support is preferably dried and reduced with hydrogen or a hydrogen containing gas. In another preferred method, the impregnated support is dried, oxidized with air or oxygen and reduced in the presence of hydrogen.

Typically, at least a portion of the metal(s) of the catalytically active metal component (a) of the catalysts of the present invention is present in a reduced state (i.e., in the metallic state). Therefore, it is normally advantageous to activate the catalyst prior to use by a reduction treatment, in the presence of hydrogen at an elevated temperature. Typically, the catalyst is treated with hydrogen at a temperature in the range of from about 75° C. to about 500° C., for about 0.5 to about 24 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume.

The present catalyst contains a catalytically effective amount of a Fischer-Tropsch metal. The amount of metal present in the catalyst may vary widely. Typically, when the catalyst includes a support, the catalyst comprises from about 1 to 50% by weight (as the metal) of the total supported metal per total weight of catalytically active metal and support, preferably from about 1 to 30% by weight, and more preferably from about 1 to 25% by weight. A Fischer-Tropsch metal may include an element selected from among a Group 8 element (e.g. Fe, Ru, and Os), a Group 9 element (e.g. Co, Rh, and Ir), a Group 10 element (e.g. Ni, Pd, and Pt), and combinations thereof. Preferably, the Fischer-Tropsch metal includes cobalt. Supported catalysts according to a preferred embodiment of the present invention may be used in the form of powders, particles, pellets, monoliths, honeycombs, packed beds, foams, and aerogels.

A catalyst prepared using a surfactant according a preferred embodiment of the present invention may include ruthenium, preferably in a minor amount. In particular, when included in a cobalt-containing catalyst, ruthenium is preferably added to the catalyst in a concentration sufficient to provide a weight ratio of elemental ruthenium: elemental cobalt of from about 0.00005:1 to about 0.25:1, preferably from about 0.0005:1 to about 0.05:1, most preferably from about 0.0005:1 to 0.01:1 (dry basis).

Alternatively, a catalyst prepared using a surfactant according a preferred embodiment of the present invention may include rhenium, preferably in a minor amount. In particular, when included in a cobalt-containing catalyst, rhenium is preferably added to the support in a concentration sufficient to provide a weight ratio of elemental rhenium: elemental cobalt of from about 0.001:1 to about 0.25:1, preferably from about 0.001:1 to about 0.05:1 (dry basis).

The catalyst may include rhenium and ruthenium. In particular, when included in a cobalt-containing catalyst, the ruthenium and rhenium are preferably added to the support in the concentrations as described above.

The catalyst may additionally or alternatively include any other suitable promoter added in suitable concentrations. The promoter is preferably a promoter for a Fischer-Tropsch catalyst, more preferably a cobalt-based catalyst. The promoter may be any known Fischer-Tropsch promoter, preferably one that improves the activity of a catalyst in the Fischer-Tropsch reaction. The promoter is preferably selected from the group consisting of the elements of Group 5 (e.g. V, Nb, and Ta), the elements of Group 6 (e.g. Cr, Mo, and W), the elements of Group 7 (e.g. Mn, Tc, and Re), the elements of Group 8, the elements of Group 9, the elements of Group 10, the elements of Group 11 (e.g. Cu, Ag, and Au), the elements of Group 12 (e.g. Zn, Cd, and Hg), the elements of Group 13 (e.g. B, Al, Ga, In, and Tl), and the elements of Group 14 (e.g. C, Si, Ge, Sn, and Pb) of the Periodic Table, more preferably from among rhenium, ruthenium, platinum, copper, silver, boron, manganese, still more preferably from among boron, copper, platinum, and silver. The weight ratio of elemental promoter to elemental Fischer-Tropsch metal, preferably cobalt, is preferably between about 0.00005:1 and about 0.5:1. Further, suitable promoters, and concentrations thereof, include those disclosed in commonly assigned U.S. Pat. No. 6,333,294, issued from U.S. patent applications Ser. No. 09/314,811, Attorney Docket Number 1856- 00800, entitled “Fischer-Tropsch Processes and Catalysts Using Promoters”, and U.S. patent application Ser. No. 09/804,271, Attorney Docket Number 1856-00803, entitled “Fischer-Tropsch Processes and Catalysts with Promoters”, and U.S. patent application Ser. No. 10/047,231, Attorney Docket Number 1856-16302, entitled “Boron Promoted Catalysts and Fischer-Tropsch Processes”, each hereby incorporated herein by reference.

The present catalyst material may be supported on any suitable support. Supports that are contemplated for use in a method for making a catalyst using a surfactant according the preferred embodiments of the present invention include silica, titania, titania/alumina, zirconia, alumina, silica, titania, titania/alumina, and the like. Further, suitable supports include those disclosed in commonly assigned U.S. Pat. No. 6,368,997, issued from U.S. patent application Ser. No. 09/314,921, Attorney Docket Number 1856-00600, entitled “Fischer-Tropsch Catalysts and Processes Using Fluorided Supports”, U.S. Pat. No. 6,365,544, issued from U.S. patent application Ser. No. 09/314,920, Attorney Docket Number 1856-00700, entitled “Fischer-Tropsch Processes and Catalysts Using Fluorided Alumina Supports”, and co-pending commonly assigned U.S. patent application Ser. No., 09/898,287 entitled “Fischer-Tropsch Processes and Catalysts Using Aluminum Borate Supports”, claiming priority to U.S. Provisional Application Serial No. 60/215,718, Attorney Docket Number 1856-08000, entitled “Fischer-Tropsch Processes and Catalysts Using Aluminum Borate Supports”. Each of the above-listed patents and patent applications is hereby incorporated herein by reference.

The catalysts of the preferred embodiments of the present invention are preferably used in a catalytic process for production of hydrocarbons, most preferably the Fischer-Tropsch process. The feed gases charged to the process of the preferred embodiment of the present invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, the feed gas stream contains hydrogen and carbon monoxide in a molar ratio of about 2:1. The feed gas may also contain carbon dioxide. The feed gas stream should contain a low concentration of compounds or elements that have a deleterious effect on the catalyst, such as poisons. For example, the feed gas may need to be pre-treated to ensure that it contains low concentrations of sulfur or nitrogen compounds such as hydrogen sulfide, ammonia and carbonyl sulfides.

The feed gas is contacted with the catalyst in a reaction zone. Mechanical arrangements of conventional design may be employed as the reaction zone including, for example, fixed bed, fluidized bed, slurry phase, slurry bubble column, reactive distillation column, or ebullating (also termed ebulliating) bed reactors, among others, may be used, or combinations thereof, in one or more stages. Accordingly, the size and physical form of the catalyst particles may vary depending on the reactor in which they are to be used.

The Fischer-Tropsch process is typically run in a continuous mode. In this mode, the gas hourly space velocity through the reaction zone typically may range from about 10 volumes/hour/volume catalyst (v/hr/v) to about 10,000 v/hr/v, preferably from about 300 v/hr/v to about 2,000 v/hr/v. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psig (653 kPa) to about 1000 psig (6994 kPa), preferably, from 80 psig (653 kPa) to about 600 psig (4237 kPa), and still more preferably, from about 140 psig (1066 kPa) to about 400 psig (2858 kPa).

The products resulting from the process will have a great range of molecular weights. Typically, the carbon number range of the product hydrocarbons will start at methane and continue to the limit observable by modem analysis, about 50 to 100 carbons per molecule. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and vapor phase products. The effluent stream of the reaction zone may be cooled to effect the condensation of additional amounts of hydrocarbons and passed into a vapor-liquid separation zone separating the liquid and vapor phase products. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following embodiments are to be construed as illustrative, and not as constraining the scope of the present invention in any way whatsoever. For example, it will be understood that while batch testing is described, a process for producing hydrocarbons may alternatively be operated in continuous mode.

### EXAMPLES

General Procedure for Catalyst Preparation

The catalyst preparation mixture resulting from contacting the catalyst material with the support material and contained in the roto-vap flask was rotated on a roto-vap for approximately 3 hours at 75° C. and 10 in. Hg vacuum. The remaining water was removed under 27 in. Hg vacuum and 75° C. The remaining solids were broken up and ground, if necessary. The solids were calcined in flowing air with the following temperature profile. The temperature was raised to 150° C. @ 5° C./min. The temperature was then maintained at 150° C. for 1 hour. Then the temperature was raised from 150° C. to 400° C. @ 3° C./min. Finally, the temperature was maintained at 400° C. for 5 hours. The calcined catalyst was allowed to cool to ambient temperature and stored in air.

General Procedure for Catalyst Activation

Prior to reaction the catalyst was reduced in flowing H₂:N₂, 1:1. The following temperature profile was used. The temperature was increased from ambient to 125° C. at 5° C./min, maintained at 125° C. for 0.5 hours; increased from 125° C. to 450° C. at 3° C./minute and maintained at 450° C. for 3 hours. The catalysts were then stored in an inert atmosphere.

General Procedure for Batch Testing

A batch screening reactor was used for catalyst testing. The catalyst was contacted in the reactor with a gas containing hydrogen and carbon monoxide in a ratio of 2:1. The reactor included a pressure vessel equipped with a gas valve, a pressure transducer and a rupture disk. The reactor was charged with catalyst while in an inert atmosphere and then pressured to reaction conditions using a synthetic blend of synthesis gas. The reaction was initiated by placing the charged reactor in a heat source. The reaction conditions included a temperature of 220° C. and an initial pressure of 600 psig. The weight percent of methane, CO, H2, and C₁ to C₄ hydrocarbon products were measured with a Gas Chromatograph. A C₅₊selectivity and a total productivity, were calculated from the measured values. The total productivity and the C₅₊selectivity were used to compute a C₅₊productivity.

Results of comparative testing are shown in Table 1. Table 1 illustrates the improved activity of a catalyst prepared using a surface active agent according to a preferred embodiment of the present invention. The CO conversion increased by 44%, the C₅₊productivity increased by 42%, and the methane make decreased by 33%.

### Example 1

### Catalyst Utilizing Surfactant

The catalyst, as prepared, had a nominal metal/promoter concentration of 20% cobalt/0.5% rhenium/1% boron supported on gamma-alumina.

The following metal salts were added to a roto-vap flask containing 40.23 g γ-Al₂O₃: 42.31 g Co(C₂H₃O₂)₂. 4H₂O, 2.89 g H₃BO₃, and 0.4936 HReO₄ solution (51% Re). A solution of 79.01 g H₂O containing approximately 0.1% Triton X-100 (a non-ionic surfactant, polyoxyethylene (10) isooctylphenyl ether) was added to the flask.

The resulting mixture was treated as described above in the general procedure for catalyst preparation. The catalyst was activated as described above before testing in the BSR reactor using the procedure as described above.

The results obtained for CO conversion, methane make and C₅₊productivity are shown in Table 1 under the heading surfactant catalyst.

### Example 2

### Comparative Catalyst

A comparative catalyst of identical nominal cobalt and promoter concentrations was prepared, using the same general preparation procedure as described above for Example 1. The actual support and metal salt amounts were as follows: 40.05 g γ-Al₂O₃, 42.25 g Co(C₂H₃O₂)₂.4H₂O, 1.54 g B₂O₃, and 0.54 g HReO₄ solution (51% Re). To this was added 50.25 g H₂O. No Triton X-100 or any other surfactant was used.

The resulting mixture was treated as described above in the general procedure for catalyst preparation. The catalyst was activated as described above before testing in the BSR reactor using the procedure as described above.

The results obtained for CO conversion, methane make (wt. % methane), and C₅₊productivity are shown in Table 1 under the heading non-surfactant catalyst.

TABLE 1

| | Non-Surfactant | Surfactant | |
|---|---|---|---|
| | Catalyst | Catalyst | Improvement |
| CO Conversion (%) | 46.65 | 67.22 | 44% |
| Methane Make (%) | 2.21 | 1.48 | 33% |
| C5+ Productivity | 102.5 | 145.9 | 42% |
| (g/hr/kg cat) | | | |

While preferred embodiments of this invention have been shown and described, modifications thereof can be made by one skilled in the art without departing from the spirit or teaching of this invention. The embodiments described herein are exemplary only and are not limiting. Many variations and modifications of the catalyst and process are possible and are within the scope of the invention. Accordingly, the scope of protection is not limited to the embodiments described herein, but is only limited by the claims that follow, the scope of which shall include all equivalents of the subject matter of the claims.

What is claimed is:

1. A method for making a catalyst comprising:
contacting a solution containing a catalyst material with a support material in the presence of a surfactant,
wherein the surfactant is selected from the group consisting of cationic surfactants and non-ionic surfactants;
drying the resulting mixture to form solids; and
calcining the dried solids.

2. The method according to claim 1 wherein the catalyst material comprises at least one Fischer-Tropsch metal.

3. The method according to claim 2 wherein the catalyst material further comprises at least one promoter.

4. The method according to claim 1 wherein the catalyst material comprises cobalt.

5. The method according to claim 4 wherein the catalyst material further comprises at least one promoter.

6. The method according to claim 5 wherein the catalyst material further comprises rhenium.

7. The method according to claim 5 wherein the catalyst material further comprises boron.

8. A method for producing hydrocarbons, comprising:
contacting a feed stream comprising hydrogen and carbon monoxide with a catalyst at conversion promoting conditions sufficient to produce an effluent stream comprising hydrocarbons;
wherein the catalyst is made by the method according to claim 1.

9. A method for making a catalyst from a support material and a catalyst material comprising:
providing a mixture of the support material and the catalyst material, wherein the catalyst material comprises an amount of Fischer-Tropsch metal catalytically active for the Fischer-Tropsch reaction;
adding a surfactant to the mixture in an amount sufficient to improve the activity of the catalyst.

10. The method according to claim 9 wherein the activity is improved by at least about 5%.

11. The method according to claim 9 wherein the activity is improved by at least about 20%.

12. The method according to claim 9 wherein the activity is improved by at least about 30%.

13. The method according to claim 1 wherein the catalyst material further comprises at least one promoter selected from the group consisting of the elements of Groups 5-14.

14. The method according to claim 13 wherein the weight ratio of elemental promoter to elemental Fischer-Tropsch metal is between about 0.00005:1 and about 0.5:1.

15. The method according to claim 9 wherein the Fischer-Tropsch metal is cobalt.

16. The method according to claim 15 wherein the cobalt is added to the support to provide a weight ratio of cobalt to support of between about 1:100 to about 1:2.

17. The method according to claim 15 wherein the catalyst material further comprises at least one promoter selected from the group consisting of elements of Groups 5-11, Group 13, and Group 14.

18. The method according to claim 17 wherein the weight ratio of elemental promoter to elemental cobalt is between about 0.00005:1 and about 0.5:1.

19. A method for producing hydrocarbons, comprising:
contacting a feed stream comprising hydrogen and carbon monoxide with a catalyst at conversion promoting conditions sufficient to produce an effluent stream comprising hydrocarbons;
wherein the catalyst is made by the method according to claim 9.

20. A method for making a catalyst from a support material and a catalyst material comprising:
providing a mixture of the support material and the catalyst material, wherein the catalyst material comprises an amount of cobalt catalytically active for the Fischer-Tropsch reaction;
adding a surfactant to the mixture in an amount sufficient to improve the activity of the catalyst by at least about 5%.

21. A method for producing hydrocarbons, comprising:
contacting a feed stream comprising hydrogen and carbon monoxide with a catalyst so as to produce an effluent stream comprising hydrocarbons;
wherein the catalyst is made by the method according to claim 20.

22. The method according to claim 20 wherein the CO conversion is improved.

23. The method according to claim 20 wherein the C₅₊ productivity is improved.

24. A method for producing hydrocarbons, comprising:
contacting a feed stream comprising hydrogen and carbon monoxide with a catalyst so as to produce an effluent stream comprising hydrocarbons;
wherein the catalyst is made by the method according to claim 20.

25. The method according to claim 23 wherein the CO conversion is improved.

26. The method according to claim 23 wherein the C₅₊ productivity is improved.
